(19) United States
(12) Patent Application Publication
(10) Pub. No.: US 2009/0182195 A1
(43) Pub. Date: Jul. 16, 2009

(54) ENDOSCOPIC GUIDE SYSTEM

(75) Inventors: Craig N. Faller, Milford, OH (US); David L. Kohn, Edgewood, KY (US)

Correspondence Address: Ethicon Endo-Surgery/Nutter, McClennen & Fish LLP, World Trade Center West, 155 Seaport Blvd., Boston, MA, 02210-2604, US

(73) Assignee: ETHICON ENDO-SURGERY, INC., Cincinnati, OH (US)

(21) Appl. No.: 11/972,840

(22) Filed: Jan. 11, 2008

(51) Int. Cl. A61B 1/01; B29C 45/00

(52) U.S. Cl. 600/114; 264/328.1

(57) ABSTRACT

Various devices for use in endoscopic surgery and methods for manufacturing the same are provided. In one embodiment, an endoscopic guide device is provided and includes an injection molded rail and a c-shaped channel defining a track extending through the rail. The channel includes a plurality of slots formed in the c-shaped channel during the injection molding process. A plurality of wings can extend from opposed sides of the c-shaped channel.

### FIELD

The present application relates to devices for use in endoscopic surgery and methods for manufacturing the same.

### BACKGROUND

The popularity of minimally invasive surgical procedures is increasing as it allows for smaller incisions which tend to reduce recovery time and complications during surgical procedures. One option that is being widely used in a wide range of procedures is endoscopic devices, which are one of the least invasive surgical methods used today as access to a surgical site can be provided through a natural body orifice or a small incision. Endoscopes are used often in a variety of endolumenal procedures (e.g., polypectomy or endoscopic mucosal resection—EMR, or endoscopic sub-mucosal dissection—ESD) or translumenal peritoneal surgeries that need access to the peritoneal cavity, including appendectomies and cholecystectomies.

The ability to manipulate a tool at a surgical site can be limited. For example, the devices and methods used to place a tool may restrict its movement relative to the surgical site, to an endoscope, or to other tools. At the same time, many endoscopic procedures require that surgical tools be positioned or used independently at the surgical site. For example, oftentimes it is desirable that an endoscope provide a view of a surgical site and/or the distal end of a surgical tool. The view of the endoscope may be limited to nearby objects within a small viewable area in front of the endoscope and require manipulation of the endoscope and/or the surgical tool in order to obtain an adequate view. A procedure may also call for the cooperative use of two or more surgical tools and may necessitate precise placement and orientation of such tools with respect to one another. For example, one tool may be employed to manipulate or grasp tissue while another tool dissects the tissue. Other problems can present themselves as the length of the devices used to place the tools at the surgical site are increased.

While current tools are effective, there is a need for improved devices for use in endoscopic surgery and for methods for forming such devices.

### SUMMARY

Various devices for use in endoscopic surgery and methods for manufacturing the same are provided. In one embodiment, an endoscopic guide device is provided and includes an injection molded rail, and a c-shaped channel defining a track extending through the rail and having a plurality of slots formed in the c-shaped channel during the injection molding process. A plurality of wings can extend from opposed sides of the c-shaped channel.

The plurality of slots formed in the c-shaped channel can have a variety of configurations. In one embodiment, the plurality of slots have an open end and a closed terminal end, with the closed terminal end being curved. The curve of the closed terminal end can have a radius in the range of 0.005 to 0.250 inches. The plurality of slots can also be spaced apart from one another along a longitudinal axis of the c-shaped channel. In addition, the c-shaped channel can be formed from a plurality of channel segments that are mated to one another.

The endoscopic guide device can also include additional features. In one embodiment, the guide device can include at least one pin coupling the channel and the plurality of wings such that the plurality of wings extend from opposed sides of the channel. The guide device can also include a sheath mated to opposed sides of the channel such that the sheath forms a lumen extending therethrough and containing the plurality of wings therein. In an exemplary embodiment, the sheath can be elastomeric.

In another embodiment, an endoscopic guide device is provided and includes a substantially c-shaped channel having a plurality of slots formed therein, and an elongated support having a plurality of wings extending from opposed sides thereof. At least one pin couples the channel and the elongated support such that the plurality of wings extend from opposed sides of the channel. In an exemplary embodiment, the c-shaped channel can include opposed side walls and a base wall connecting the side walls. Each slot can extend through the opposed side walls and the base wall of the channel. The plurality of slots can also be spaced apart from one another along a longitudinal axis of the c-shaped channel, and can have an open end and a closed terminal end. The closed terminal end can be curved, and the curve can have a radius in the range of 0.005 to 0.250 inches.

The endoscopic guide device can optionally include additional features, such as a sheath mated to opposed sides of the channel such that the sheath forms a lumen extending therethrough and containing the plurality of wings therein. In an exemplary embodiment, the sheath can be elastomeric. The c-shaped channel can also be formed in a variety of ways. In one embodiment, the c-shaped channel is formed from a plurality of channel segments that are mated to one another. Each channel segment can include first and second terminal ends having projections formed therein and configured to interlock with projections formed on an adjacent channel segment to be mated thereto.

Methods for manufacturing an endoscopic guide device are also provided, and in one embodiment the method can include injection molding an elongated rail having a track formed therein and extending along a longitudinal axis, and a plurality of slots extending across the track generally transverse to the longitudinal axis. In one embodiment, the elongated rail can be injection molded in a plurality of channel segments that are mated to one another to form the elongated rail, and the plurality of slots extending across the track can have an open end and a closed terminal end, with the closed terminal end being curved. The method further includes injection molding a support member extending having a plurality of opposed wings extending from opposed sides thereof, and mating the elongated rail and the support member. In one embodiment, mating the elongated rail and the support member can include coupling one or more pins between one or more corresponding first and second mating elements formed in the elongated rail and the support member to couple the support member to the elongated rail. In an exemplary embodiment, the method can also include mating a sheath having a lumen therein to the elongated rail such that the lumen houses the channel and the winged members therein.

### BRIEF DESCRIPTION OF THE DRAWINGS

The invention will be more fully understood from the following detailed description taken in conjunction with the accompanying drawings, in which:

FIG. 1 is a perspective view of one embodiment of an endoscopic guide device;

FIG. 2 is an enlarged perspective view of a proximal end of the endoscopic guide device of FIG. 1;

FIG. 3 is an enlarged partially transparent perspective view of a distal end of the endoscopic guide device of FIG. 1;

FIG. 4 is a perspective view of a channel of the endoscopic guide device of FIG. 1;

FIG. 5 is a bottom view of a channel of the endoscopic guide device of FIG. 1;

FIG. 6 is another perspective view of a channel of the endoscopic guide device of FIG. 1;

FIG. 7 is a perspective view of a channel of the endoscopic guide device of FIG. 1 showing two channel segments detached from one another; and

FIG. 8 is a perspective view of an elongated support of the endoscopic guide device of FIG. 1.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary devices for use in endoscopic surgery and methods of manufacturing the same are provided. In an exemplary embodiment, endoscopic guide devices are provided which can include a channel and an elongated support coupled thereto. The channel can be configured to mate to an elongated tubular member to allow the tubular member to extend along the guide device, thus allowing various tools and/or material to be passed through the tubular member for delivery to a surgical site. In an exemplary embodiment, the channel and the elongated support are formed using an injection molding process in order to provide improved flexibility features and to minimize the occurrence of fractures formed in the channel and elongated support while flexing during use.

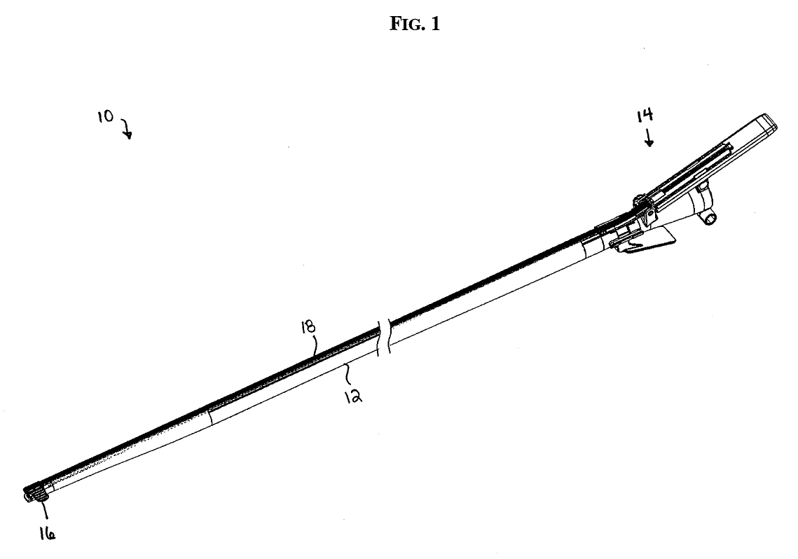

While the present invention can be used with a variety of endoscopic guide systems, FIG. 1 illustrates one exemplary embodiment of an endoscopic guide device 10 that can be used for delivering various instruments, materials, and/or irrigation along an external length of an endoscope. As shown, the device 10 generally includes a flexible sheath 12 that is sized and shaped to receive an endoscope insertion portion or other device therein. The device 10 can also include a handle 14 located on a proximal end thereof and a tip 16 mated to a distal end thereof. The endoscopic guide device 10 can further include a channel 18 having a flexible track on a superior surface thereof that extends along a portion(s) or the entire length of the sheath 12 from the handle 14 to the tip 16. In one embodiment, a distal end of the channel 18 can extend a distance beyond a distal end of the sheath 12 to allow the channel 18 to mate to the tip 16, and a proximal end of the channel 18 can extend a distance beyond the proximal end of the sheath 12 to mate with the handle 14. An elongated support 20 can be coupled to the channel 18 and it can have wings 22 extending therefrom. In use, the channel 18 can be configured to mate to an elongated tubular member, such as an accessory channel, for receiving various tools and/or material, i.e., accessories, therethrough. In one exemplary embodiment, the channel 18 can guide the accessory to and from the site of therapy alongside the insertion portion of the endoscope after the endoscope has been placed within the patient's anatomy.

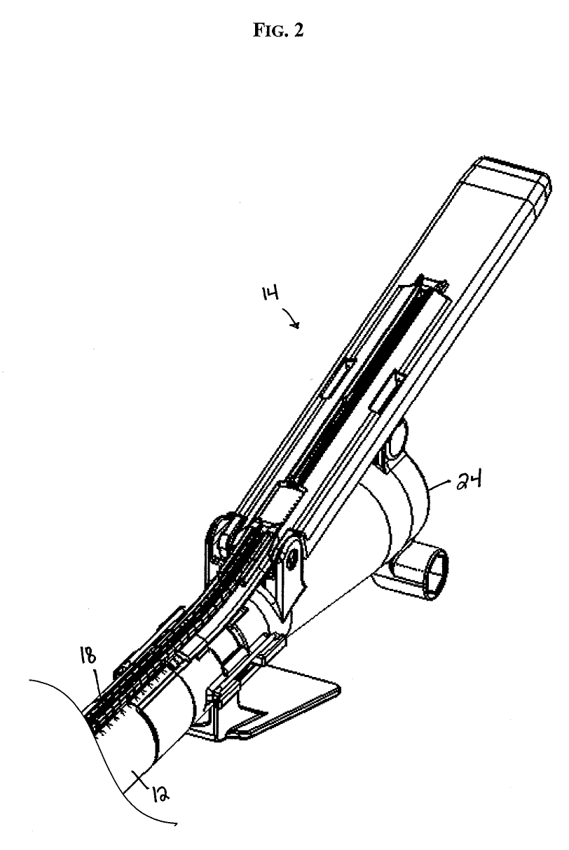

FIG. 2 shows the proximal end of the guide device 10 in more detail, and illustrates the handle 14 that is coupled to a proximal end of the device 10. The handle 14 can have any shape and size but it is preferably adapted to facilitate grasping and/or manipulation of the system 10, and optionally loading of an accessory channel onto the system 10. Additionally, the handle 14 can be used to affix the device 10 to a proximal end of an endoscope to maintain the axial and radial position of the device 10 on the endoscope as well as prevent the device 10 from buckling on the endoscope during accessory device insertion and/or removal. As shown in FIGS. 1 and 2, the handle 14 has a generally elongated configuration. The handle 14 can also include an opening 24 formed at a proximal end thereof. The opening 24 is adapted to allow an endoscope insertion portion to be inserted therethrough and into the sheath 12. While a handle is illustrated, a person skilled in the art will appreciate that the device need not include a handle.

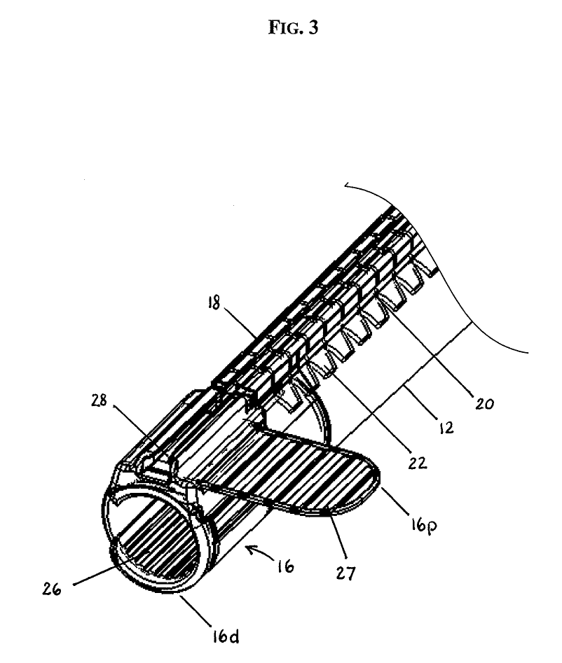

FIG. 3 shows the distal end of the guide device 10 in more detail, and illustrates the tip 16 coupled to a distal end of the channel 18. The tip 16 can have a variety of configurations, but in the illustrated embodiment it has a generally cylindrical body portion with proximal and distal ends 16p, 16d and a central bore 26 extending therethrough for receiving the distal end of an endoscope that is passed through the opening 24 in the handle and through the sheath 12. The tip 16 can also include a locking mechanism, such as a tab 27, extending radially therearound for locking the endoscope to the tip 16. Locking the tip 16 to a distal end of the endoscope can maintain the axial and radial position of the device 10 on the endoscope as well as prevent the device 10 from buckling on the endoscope during accessory device insertion and/or removal. The tip 16 can include various other features as well. For example, as shown, a passage 28 is formed in a proximal portion of a superior surface of the tip 16 such that the passage 28 extends from the proximal end and terminates proximal to the distal end. The passage 28 can be insert molded or connected through secondary operations such as heat staking or ultrasonic welding, and it can fixedly mate to the distal end of the channel 18 that extends along the sheath 12. The passage 28 is configured to receive a portion of an accessory channel mated to the guide device 10 to limit insertion of the accessory device. While a tip is illustrated, a person skilled in the art will appreciate that the device need not include a tip.

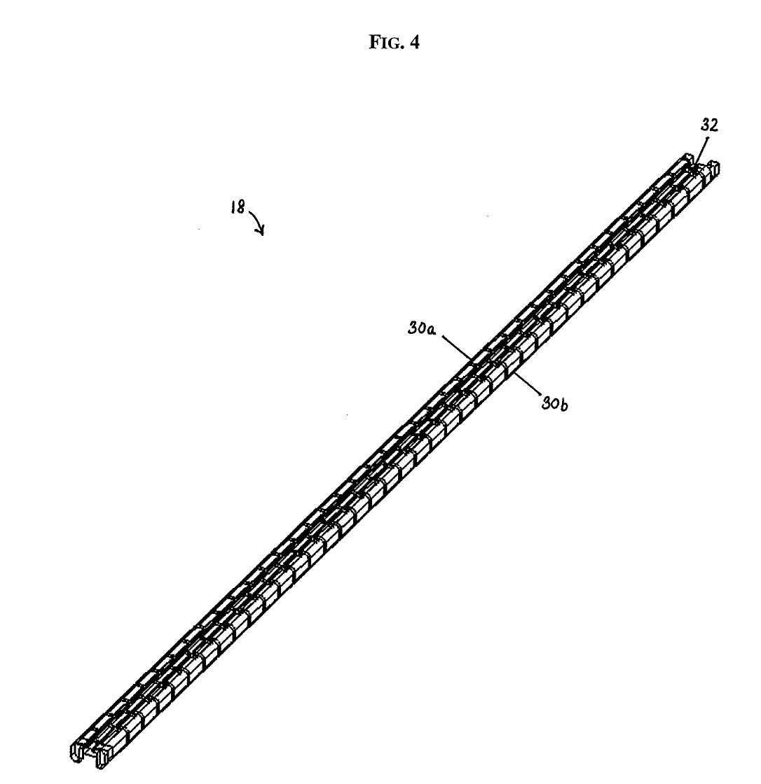

As indicated above, the device 10 also includes a channel extending along all or a portion thereof and adapted to couple to an elongated tubular member, such as an accessory device, thus allowing various instruments, materials, and/or irrigation to be delivered through the accessory device to a surgical site. Such a configuration is particularly advantageous as it eliminates the need to remove and re-introduce the endoscope, and it also provides a secondary pathway to the surgical site. In one exemplary embodiment, as shown in FIGS. 4-7, the channel 18 has a generally elongated, c-shaped configuration with opposed side walls 30a, 30b and a base wall 32 connecting the side walls 30a, 30b.

The channel 18 thus defines a track extending therethrough for receiving a complementary mating feature formed on an accessory device. For example, the c-shaped channel 18 can be configured to slidably receive a T-shaped rail on an accessory device. A person skilled in the art will appreciate that the channel 18 can have various other configurations to allow mating with an accessory device.

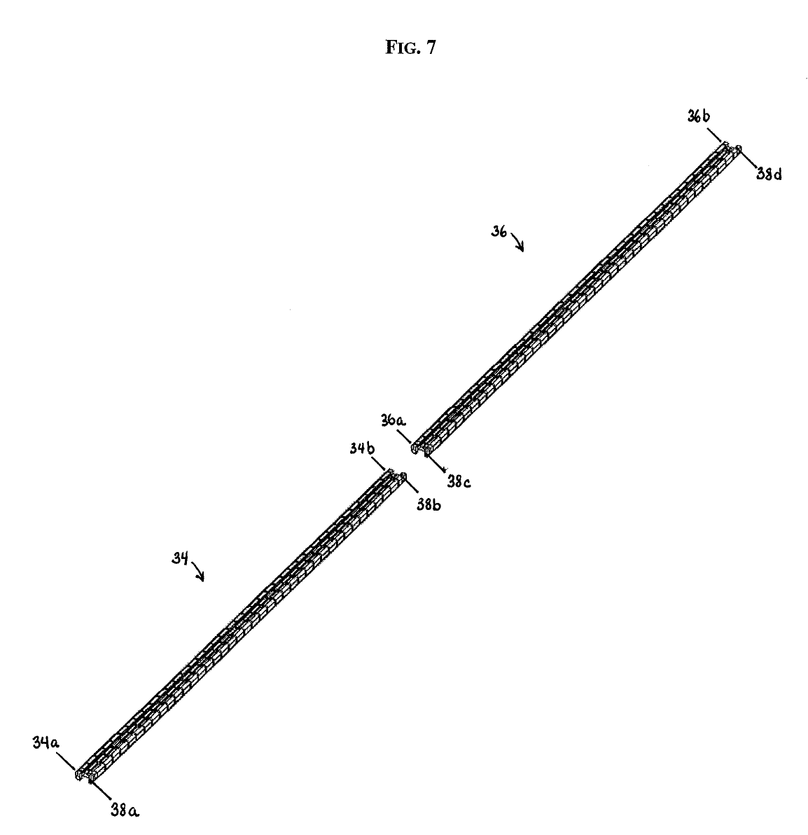

The channel 18 can be formed from a single channel segment, as shown in FIG. 4, or it can be formed from a plurality of channel segments that can be coupled together in a variety of ways. For example, if the channel is formed from a plurality of channel segments 34, 36, as shown in FIG. 7, first and second terminal ends 34a, 34b, 36a, 36b of each channel segment 34, 36 can include first and second projections 38a, b, c, d formed therein. For example, the projection 38b formed on the second terminal end 34b of the first channel segment 34 can be configured to interlock with the projection 38c formed on the adjacent first terminal end 36a of the second channel segment 36 to be mated thereto. The projections 38 can be in the formed of first and second fingers extending from the ends of the channel segments 34, 36 that are adapted to mate to the fingers of an adjacent projection 38. A person skilled in the art will appreciate that the ends can have any configuration that allows them to mate to each other to couple adjacent channel segments together and provide continuity between the channel segments. In an exemplary embodiment, however, the ends are configured to conform to the channel so that it does not interfere with slidable mating of an accessory device to the channel. Moreover, a person skilled in the art will appreciate that any number of channel segments can be used to facilitate the desired flexibility and length of the channel, and that any technique can be used to couple adjacent channel segments.

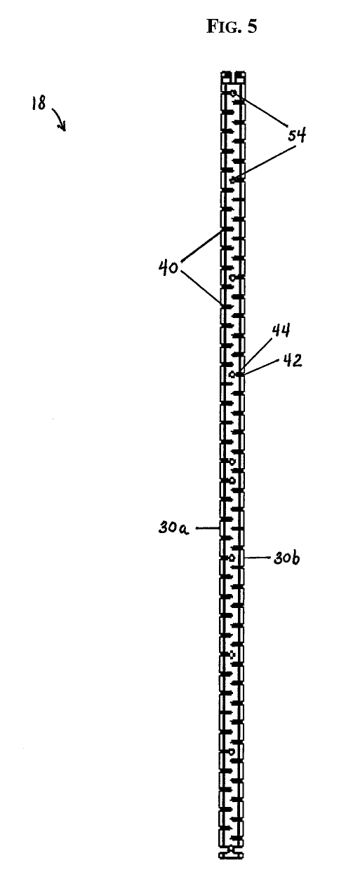

In an exemplary embodiment, as indicated above, the channel 18 is adapted to be inserted into a patient's alimentary tract or other natural orifice. Thus, at least a portion of the channel 18 can be semi-flexible or flexible to allow insertion through a tortuous lumen. For example, the channel 18 can include features formed thereon to improve flexibility of the channel 18 and reduce its bending stiffness. In one exemplary embodiment, the channel 18 can include a plurality of slots 40, shown in FIG. 5, that extend through the opposed side walls 30a, 30b and optionally the base wall 32 of the channel 18. The slots 40 can have an open end 42 located in the sidewalls 30a, 30b and a closed terminal end 44 located in the base wall 32. The slots 40 can be formed in the channel 18 in a variety of ways, but in an exemplary embodiment the slots 40 are generally transverse, and more preferably perpendicular, to the longitudinal axis of the channel 18. The channel 18 can include any number of slots 40, and the slots 40 can be positioned in a variety of ways along the length of the channel 18. For example, the slots 40 can be uniformly or non-uniformly spaced apart from one another along a longitudinal axis of the channel 18. The slots 40 can also be aligned to allow a single slot 40 to extend through both sidewalls 30a, 30b, but preferably the slots 40 are offset from one another, as shown in FIG. 5. For example, a first slot can extend into only one sidewall 30a and partially into the base wall 32. An adjacent slot can be longitudinally offset from the first slot and can extend into the other sidewall 30b and partially into the base wall 32. The remainder of the slots can continue to follow such an alternating pattern. A person skilled in the art will appreciate that the slots 40 can be positioned along the channel 18 in any configuration that facilitate the desired flexibility of the channel 18.

The closed terminal end 44 of each slot 40 can also have a variety of configurations, but preferably the closed terminal end 44 is curved. In an exemplary embodiment, the curve has a radius in the range of 0.005 to 0.250 inches. A person skilled in the art will appreciate, however, that the desired radius can be sized to match the desired slot width needed to achieve the flexibility that is required. In order to form the curve in the closed terminal end 44 of the slots 40, an injection molding process is used. This allows the curve to be formed during the process of molding the channel 18. This is an improvement over an extrusion process, in which the slots are cut into the channel after the channel is formed. The slot cutting process causes the material forming the channel to push apart to form the slot, resulting in locations of high stress where fractures can occur after repeated flexing of the assembly. The curve formed during the injection molding process, as described in more detail below, is advantageous as the curve helps to prevent stress fractures from forming in the channel 18 during flexion of the channel 18 in use.

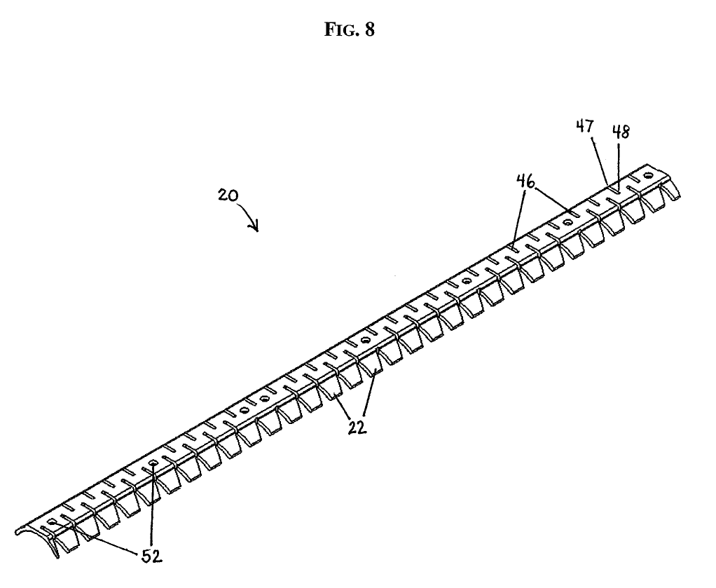

As further shown in FIG. 8, the channel 18 can also include an elongated support 20 having a plurality of wings 22 extending from opposed sides. The elongated support 20 can be configured to allow a sheath or other member to be mated to the channel 18 for receiving an endoscope. The elongated support 20 can also provide structural integrity at the joints of the channel segments when the channel 18 is formed from a plurality of segments. In one exemplary embodiment, the elongated support 20 has a generally elongated shape, and is preferably flexible or semi-flexible as it is adapted to be inserted into a patient's alimentary tract or other natural orifice. As shown in FIGS. 1-3, the elongated support 20 extends from the tip 16 to the handle 12 of the endoscopic guide device 10. The length of the elongated support 20 can vary depending on the intended use of the device 10, but in the illustrated embodiment the elongated support 20 has an elongated length that is adapted for use in the patient's alimentary tract, thus allowing the distal end of the elongated support 20 to reach a surgical site to which instruments and materials are being delivered. In order to render the elongated support 20 flexible, the elongated support 20 can include slots 46 formed therein to increase flexibility of the elongated support 20. As with the slots 40 in the channel 18, the slots 46 in the elongated support 20 can include an open end 47 and a closed terminal end 48 that is preferably curved. In an exemplary embodiment, the slots 46 extend transverse, and more preferably perpendicular, to the axis of the elongated support 20, and they are spaced longitudinally apart from one another along the length of the elongated support 20. Each slot 46 can also extend only partially across the width of the elongated support 20. Moreover, similar to the slots 40 in the channel 18, opposed slots 46 in the elongated support 20 can be longitudinally offset from one another. As a result of the slots 46, the elongated support 20 can include wings 22 extending longitudinally therealong on opposed sides thereof. The wings 22 can extend from the elongated support 20, for example, at an angle to allow the wings 22 to seat around the curvature of an endoscope that is inserted through the sheath 12. Thus, a person skilled in the art will appreciate that the wings 22 can extend from the elongated support at any angle that allows the wings 22 to sit around an endoscope or other device and provide stability to the channel 18 on the outside surface of the endoscope insertion portion. In use, the wings 22, which are separated by the slots 46, allow for increased flexibility. A person skilled in the art will appreciate that the slots 46 and the wings 22 can be formed along various portions of the elongated support 20 in any configuration, and that any number of slots 46 and wings 22 can be formed in the elongated support 20.

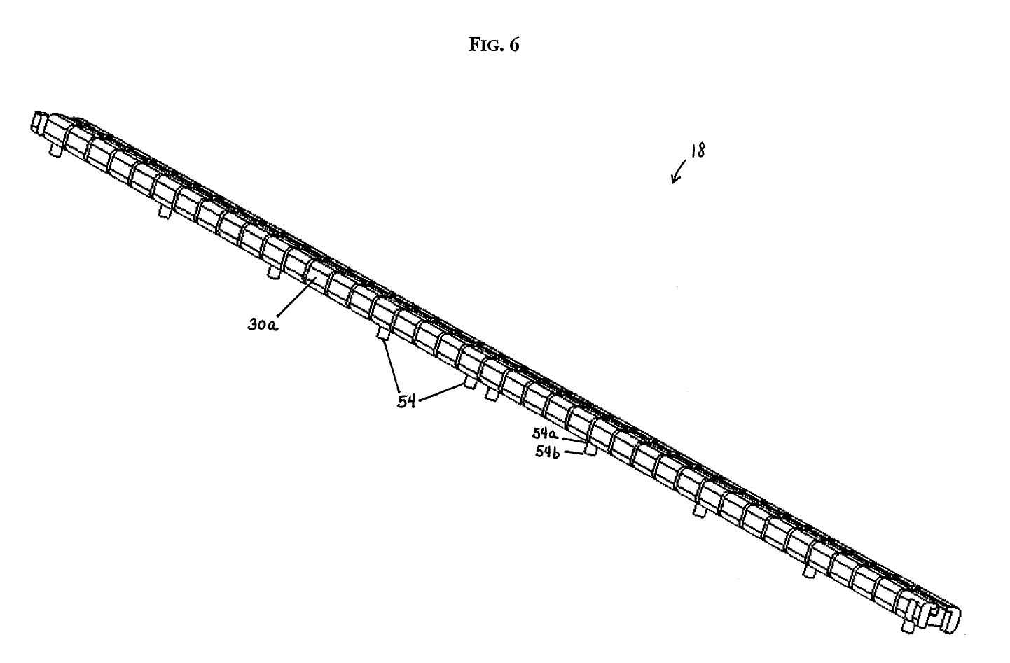

As indicated above, the elongated support 20 can mate to the channel 18. Various techniques can be utilized to couple the channel 18 and the elongated support 20 together. In one embodiment, as shown in FIGS. 6, 7, and 9, a plurality of pins 54 can be formed on the channel 18 and a plurality of bores 52 can be formed in the elongated support 20. The plurality of pins 54 can be used to couple the channel 18 and the elongated support 20, as shown in FIGS. 6, 7, and 9. The bores 52 can extend through the elongated support 20 and they can be sized and shaped to receive the pins 54 therein. The pins 54 can be in the form of elongated cylindrical members having a first end 54a adapted to extend beyond a lower surface of the channel 18 and a second end 54b adapted to sit within and extend beyond the bores 52 formed in the elongated support 20. In one exemplary embodiment, the second end 54b of the pins 54 can be used to form heads using a secondary operation, for example, ultrasonic welding or heat staking, to couple the channel 18 and the elongated support 20. The channel 18 can include any number of pins 54 and the elongated support 20 can include any number of bores 52, and the pins 54 and the bores 52 can be positioned in a variety of ways along the length of the channel 18 and the elongated support 20 to facilitate coupling between the channel 18 and the elongated support 20. For example, the pins 54 and the bores 52 can be uniformly or non-uniformly spaced apart from one another along a longitudinal axis of the channel 18 and the elongated support 20. In one embodiment, the corresponding pins 54 and bores 52 formed in the channel 18 and the elongated support 20 can be positioned in such a way that allows the channel 18 and the elongated support 20 to be coupled together in only one configuration. For example, the pins 54 and the bores 52 can be positioned along the length of the channel 18 and the elongated support 20 so that when the channel 18 and the elongated support 20 are positioned adjacent to each other, the pins 54 and the bores 52 line up and allow the channel 18 and the elongated support 20 to be coupled together in a single configuration. This can be advantageous as it can help to prevent incorrect assembly of the channel 18 and the elongated support 20. The use of the bores 52 and the pins 54 described above allow for a greater degree of flexibility between the channel 18 and the elongated support 20 as the channel 18 and the elongated support 20 are coupled together only at the locations of the bores 52 and the pins 54 that are received therein. For example, in use, one of the channel 18 and the elongated support 20 can remain relatively straight while the other of the channel 18 and the elongated support 20 can flex as necessary between the locations of the bores 52 and the pins 54. A person skilled in the art will appreciate, however, that various mating elements and techniques known in the art can be used to couple the channel 18 and the elongated support 20. Moreover, a person skilled in the art will appreciate that the pins 54 can be formed on the elongated support 20 and the bores 52 can be formed in the channel 18. In addition, the channel and the elongated support can be manufactured as a single piece, as will be described in more detail below.

As described above, the device 10 can also include a sheath 12 that can be mated to opposed sides of the channel 18 such that the sheath 12 forms a lumen extending therethrough. In one embodiment, the sheath 12 can protect the patient's anatomy from exposed edges and pinch points that can be created by the plurality of slots. In one exemplary embodiment, the sheath 12 can be coupled to a portion of the channel 18 in such a way as to contain the wings 22 on the elongated support 20 therein. The lumen extending through the sheath 12 can be adapted, as described above, to receive an endoscope insertion portion therein. The sheath can be rigid or flexible, but in the preferred embodiment the sheath 12 is flexible to allow for increased flexibility of the channel 18 to which the sheath 12 is mated. For example, the sheath 12 can be formed from an elastomeric material. A person skilled in the art will appreciate, however, that the sheath 12 can be formed from any material that gives the sheath 12 flexibility. For example, the sheath 12 can formed from materials such as polyolefins, urethanes, silicones, polypropylene, and polyethylene.

Methods of manufacturing the components of the system are also provided. In one embodiment, the channel 18 and elongated support 20 are formed using an injection molding process, as indicated above. The channel 18 and the elongated support 20 can be made from any injection grade material, such as polypropylene, polyethylene, or a blend containing polypropylene or polyethylene. Using an injection molding process, the flexibility features, namely the slots 40, 46, can be molded into the channel 18 and the elongated support 20 during manufacturing. This allows the slots 40, 46 to have more consistent dimensional properties and it allows the closed terminal ends 44, 48 of the slots 40, 44 to have a curve with a radius. The curvature of the closed terminal end 44, 48 can act as points of stress relief during flexion of the channel 18 and the elongated support 20, thus reducing propagation of fractures in the channel 18 and the elongated support 20 as the slots 40, 46 have substantially no sharp edges where fractures can occur.

As described above, the channel 18 can be formed from one single channel. This can be achieved using an overmolding injection molding technique that molds together smaller channel segments during the manufacturing process, for example, by indexing previously molded components and shutting off the mold on one end of the previously molded component. The channel 18 can also be formed from a plurality of channel segments. In one exemplary embodiment, the channel segments can be molded into segments having a length up to 6 inches. A person skilled in the art will appreciate that the length of the channel segments can vary depending on the type of manufacturing process that is employed.

Once formed, the channel 18 and the elongated support 20 can be mated together using the pins 54. For example, the channel 18 and the elongated support 20 can be lined up based on the locations of the corresponding pins 54 and bores 52. The bores 52 receive the second ends 54b of the pins 54. The pins 54 can be mated to the bores 52 of the elongated support 20 in a variety of ways. For example, an ultrasonic welder can be used to create vibration that can melt a portion of the second ends 54b of the pins 54. This creates flattened heads on the second ends 54b of the pins 54 that provides mating of the pins 54 to the elongated support 20. A person skilled in the art will appreciate that the pins 54 can be mated to the elongated support 20 in a variety of ways, such as features formed on the pins 54 that can mate the elongated support 20 without the need for the heat melt process described above. In addition, a person skilled in the art will appreciate that the pins 54 can be separate components, and the first and second ends 54a, 54b of the pins 54 can be mated to both the channel 18 and the elongated support 20.

Various techniques can also be used to couple the sheath 12 to the channel 18. In one exemplary embodiment, the sheath 12 can be in the form of an elongated length of sheet material. The two elongated ends of the material can be melted into the material of the channel 18 around the wings 22 on the elongated support 20 to form a sheath 12 having a lumen that extends therethrough. For example, an ultrasonic welder can be used to create vibration that can melt the elongated ends of the material of the sheath 12 into the material of the channel 18. Other joining methods may include, but are not limited to, heat sealing, mechanical means, and adhesives. In another exemplary embodiment, the sheath 12 can be in the form of an extruded tubular length of material. The tip 16, the channel 18 with elongated support 20, and a distal end of the handle 14 can be inserted through the tubular sheath material and the sheath material can be melted into the materials of the tip 16, the channel 18, and the distal end of the handle 14. The tubular sheath material that is preventing access of an accessory into the channel 18 can then be removed. For example, an ultrasonic welder can be used to create vibration that can melt the material of the tubular sheath 12 into the materials of the tip 16, the channel 18, and the distal end of the handle 14. Other joining methods may include, but are not limited, to heat sealing, mechanical means, and adhesives.

The devices disclosed herein can be designed to be disposed of after a single use, or they can be designed to be used multiple times. In the latter case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present invention.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag, or combination plastic and TYVEK bag. The container and instrument are then placed in a field of radiation, such as gamma radiation, x-rays, or high-energy electrons, or ethylene oxide gas that can penetrate the container. The radiation or gas kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam., or chemical means

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A endoscopic guide device, comprising:
an injection molded rail;
a c-shaped channel defining a track extending through the rail and having a plurality of slots formed in the c-shaped channel during the injection molding process; and
a plurality of wings extending from opposed sides of the c-shaped channel.

2. The device of claim 1, further comprising at least one pin coupling the channel and the plurality of wings such that the plurality of wings extend from opposed sides of the channel.

3. The device of claim 1, further comprising a sheath mated to opposed sides of the channel such that the sheath forms a lumen extending therethrough and containing the plurality of wings therein.

4. The device of claim 3, wherein the sheath is elastomeric.

5. The device of claim 1, wherein the plurality of slots have an open end and a closed terminal end, the closed terminal end being curved.

6. The device of claim 5, wherein the curve has a radius in the range of 0.005 to 0.250 inches.

7. The device of claim 5, wherein the plurality of slots are spaced apart from one another along a longitudinal axis of the c-shaped channel.

8. The device of claim 1, wherein the c-shaped channel is formed from a plurality of channel segments that are mated to one another.

9. A endoscopic guide device, comprising:
a substantially c-shaped channel having a plurality of slots formed therein;
an elongated support having a plurality of wings extending from opposed sides thereof, and
at least one pin coupling the channel and the elongated support such that the plurality of wings extend from opposed sides of the channel.

10. The device of claim 9, further comprising a sheath mated to opposed sides of the channel such that the sheath forms a lumen extending therethrough and containing the plurality of wings therein.

11. The device of claim 10, wherein the sheath is elastomeric.

12. The device of claim 9, wherein the c-shaped channel includes opposed side walls and a base wall connecting the side walls, and wherein each slot extends through the opposed side walls and the base wall.

13. The device of claim 12, wherein the plurality of slots are spaced apart from one another along a longitudinal axis of the c-shaped channel.

14. The device of claim 9, wherein the c-shaped channel is formed from a plurality of channel segments that are mated to one another.

15. The device of claim 14, wherein each channel segment includes first and second terminal ends having projections formed therein and configured to interlock with projections formed on an adjacent channel segment to be mated thereto.

16. The device of claim 9, wherein the plurality of slots have an open end and a closed terminal end, the closed terminal end being curved.

17. The device of claim 16, wherein the curve has a radius in the range of 0.005 to 0.250 inches.

18. A method of manufacturing an endoscopic guide device, comprising:
injection molding an elongated rail having a track formed therein and extending along a longitudinal axis, and a plurality of slots extending across the track generally transverse to the longitudinal axis;
injection molding a support member extending having a plurality of opposed wings extending from opposed sides thereof, and
mating the elongated rail and the support member.

19. The method of claim 18, wherein the elongated rail is injection molded in a plurality of channel segments that are mated to one another to form the elongated rail.

20. The method of claim 18, wherein mating the elongated rail and the support member includes coupling one or more pins between one or more corresponding first and second mating elements formed in the elongated rail and the support member to couple the support member to the elongated rail.

21. The device of claim 18, wherein the plurality of slots extending across the track have an open end and a closed terminal end, the closed terminal end being curved.

22. The method of claim 18, further comprising mating a sheath having a lumen therein to the elongated rail such that the lumen houses the channel and the winged members therein.
